(19) United States
(12) Patent Application Publication
(10) Pub. No.: US 2008/0154043 A1
(43) Pub. Date: Jun. 26, 2008

(54) PROCESS FOR THE MANUFACTURE OF 7-OXA-BICYCLO DERIVATIVES

(76) Inventors: Paul Spurr, Riehen (CH); Beat Wirz, Reinach (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/956,362

(22) Filed: Dec. 14, 2007

(30) Foreign Application Priority Data
Dec. 22, 2006 (EP) 06126982.5

(51) Int. Cl. C07D 493/08; C12P 17/18

(52) U.S. Cl. 548/159; 549/463; 435/119

(57) ABSTRACT

The present invention relates to a process for the manufacture of the 7-oxabicyclo derivative of the formula I

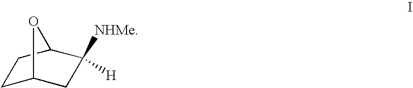

### PRIORITY TO RELATED APPLICATION(S)

This application claims the benefit of European Patent Application No. 06126982.5, filed Dec. 22, 2006, which is hereby incorporated by reference in its entirety.

### BACKGROUND OF THE INVENTION

Compounds of formula I are important intermediates for the preparation of a number of pharmaceutically active substances, for example for the preparation of adenosine receptor ligands of formula

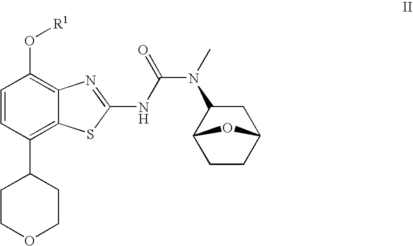

wherein R¹ is C₁₋₆-alkyl.

The adenosine receptor ligands of formula II have a good affinity to the A_{2A}-receptor and a high selectivity to the A₁- and A₃ receptors. Methods for the manufacture of compounds of formula II are described in WO05/000842 and WO01/97786.

Compounds according to formula I can be prepared for example by a Diels Alder reaction according to the method described in J. Het. Chem. 1972, 561-568. This method, however, yields racemates which require lengthy separation procedures. Therefore there exists a need for a short and cost effective method for the manufacture of compounds of formula I.

### SUMMARY OF THE INVENTION

The present invention provides a process for the manufacture of 7-oxa-bicyclo derivatives of formula

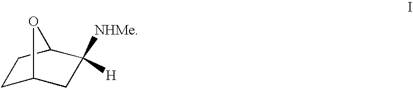

### DETAILED DESCRIPTION OF THE INVENTION

According to the invention, compounds of formula I can be prepared according to the method depicted in Scheme 1.

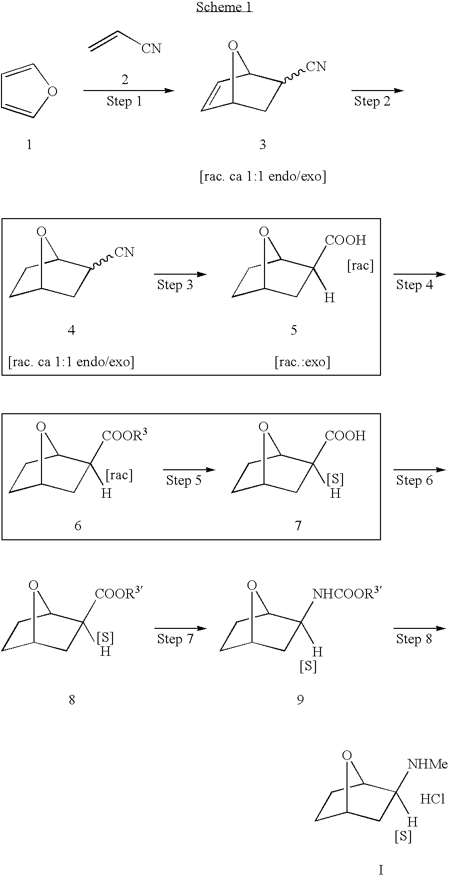

wherein R³ and R³ independently of each other are C₁-C₆-alkyl.

In the process according to the invention, a racemic exo/endo-mixture of 7-oxa-bicyclo[2.2.1]hept-5-ane carbonitrile is prepared by Diels Alder addition of acrylonitrile to furan and subsequent hydrogenation. The cyano group of compound 4 was hydrolyzed giving exclusively the exo-acid, which is then esterified. The lengthy separation process of the enantiomers can be avoided by enzymatic resolution of the ester 6 by the process according to the invention. The enzymatic racemic resolution of a similar compound, a 7-oxa-bicyclo[2.2.1]hept-5-ene-2-exo-carboxylic acid ester, has been described in the literature. The respective racemic methyl ester was resolved using lipase from Candida rugosa as described in L. L. Kissling et al.; Tetrahedron Lett. 1996, 37(49), 8853-8856 and the respective racemic ethyl ester by lipase from Candida antarctica, form B (Chirazyme L-2) as described in U.S. Pat. No. 6,403,824. In spite of the modest selectivities (E<15; for the ‘enantiomeric ratio’ E, an indicator for the enzyme selectivity, see C. J. Sih et al.; J. Am. Chem. Soc. 1982, 104, 7294-7299) observed with these enzymes, they were useful to prepare the retained (R)-unsaturated ester analogue of 6 in good enantiomeric excess (92-93% ee). However, the selectivity of these enzymes towards the exo-saturated racemate of formula 6 was only modest so unacceptable, even more as the formed [S]-acid 7 was the target enantiomer. To obtain the desired enantiomer in high enantiomeric excess, an E>100 is usually required (cf. C. J. Sih et al.; J. Am. Chem. Soc. 1982, 104, 7294-7299) and should be, for technical and economic reasons, obtained in >97% ee.

The enzyme Candida antarctica form A, a commercial source of which is Novocor AD L (Novozymes; Denmark) was identified as a suitable catalyst. This enzyme displays a high selectivity towards racemic ester 6, particularly in combination with longer alkoxy moieties (E>>100). Preferred are the butyl and pentyl esters, which can be converted at technically relevant substrate concentrations. At higher substrate concentrations, the enzyme activity turned out to be supported by using stronger phosphate buffer (cf. Example 5b), which in turn allowed the reaction to be carried out at lower temperature (within reasonable time) thus enhancing enzyme selectivity. Since the enzyme does not act on the endo-ester, which is present in only a low amount in the substrate, the endo-form can be easily removed by this step. The enantiomerically pure (S)-acid 7 was then re-esterified according to methods known in the art.

The ester 8 was reacted with hydrazine hydrate to form the acyl hydrazide which was treated with nitrous acid to form the acyl azide, thermal rearrangement thereof in the presence of an alcohol such as methanol, propanol or butanol produced the carbamate 9. Finally the carbamate is reduced in the presence of lithium aluminium hydride to form the amine of formula I.

In more detail, the process according to the invention is carried out as follows:

Step 1: Acrylonitrile was added to furan in the presence of a catalytic amount of ZnCl₂ which yields 7-oxabicyclo[2.2.1]hept-5-ene-2-carbonitrile in a racemic 1:1 mixture of exo/endo-isomers.

Step 2: Catalytic reduction of the double bond of compound 3 in the presence of a metal catalyst, such as Pd/C, yields the racemic endo/exo 7-oxabicyclo[2.2.1]heptane-2-carbonitrile 4. This reaction is described in Synlett 1996, 703-704.

Step 3: 7-Oxabicyclo[2.2.1]heptane-2-carbonitrile 4 was hydrolyzed in the presence of a strong base, such as potassium hydroxide, in an appropriate solvent, ideally in water or an alcohol, such as ethanol forming only the corresponding exo-carboxylic acid 5.

Step 4: The acid 5 is esterified by methods known in the art, for example by generating an acid chloride which is reacted with an appropriate hydroxyalkane yielding the racemic exo-derivative.

Step 5: The racemic kinetic resolution of the racemic exo-ester 6 was carried out by hydrolysis in the presence of enzyme, particularly in the presence of a lipase from Candida antarctica form A, a commercial source of which is Novocor AD L (Novozymes; Denmark). The desired (1R,2S,4S)-enantiomer is obtained as the acid 7 which is isolated conventionally by repeated extraction with an organic solvent at different pH. Optionally, the acid can be further optically enriched by means of crystallization.

Step 6: The esterification of the enantiomerically pure acid 7 is carried out according to methods known in the art, for example by acid catalyzed reaction with the corresponding alcohol, such as methanol, ethanol or propanol, to form the ester 8.

Step 7: The ester 8 was transformed into the carbamic acid ester 9 by a Curtius rearrangement, i.e. by reaction with hydrazine hydrate and subsequently with nitrous acid to form the acyl azide which is rearranged into the carbamic acid ester 9 in the presence of an alkylalcohol, as depicted in Scheme 2, the intermediates are not isolated.

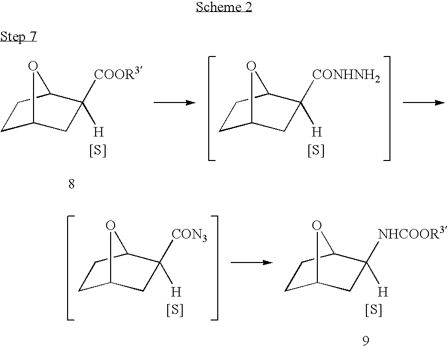

wherein R³ is C₁-C₆-alkyl.

Step 8: Finally the carbamate 9 is reduced according to methods known in the art, such as lithium aluminium hydride to form the amine of formula I.

The process according to the invention consists thus in
a) the preparation of

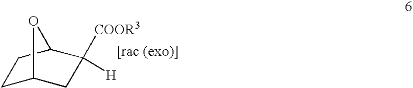

wherein R³ is C₁-C₆-alkyl

by addition of acrylonitrile to furan in the presence of a catalytic amount of ZnCl₂ to form 7-oxabicyclo[2.2.1] hept-5-ene-2-carbonitrile as a racemic 1:1 mixture of exo/endo-isomers followed by catalytic reduction of the double bond in the presence of a metal catalyst, hydrolysis of the cyano-group and esterification of the racemic exo-acid via the reaction of the acid chloride and an alkylalcohol, particularly butanol;
b) kinetic resolution of the racemic exo-ester 6 by enzymatic hydrolysis in the presence of enzyme, particularly in the presence of a lipase from Candida antarctica form A, a commercial source of which is Novocor AD L (Novozymes; Denmark), emulsified in an aqueous buffer around neutral pH, preferably in a medium wherein the phosphate buffer is employed at higher concentration in combination with lower temperature, to form the (S)-acid 7 and the (R)-ester 6-[R].

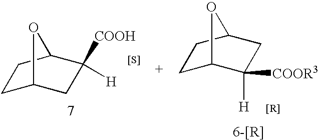

wherein R³ is as defined above

which are separated by extraction;
c) esterification of the (S)-acid 7 and subsequent reaction with hydrazine hydrate and nitrous acid followed by thermal rearrangement in the presence of an alcohol to form the carbamic acid ester 9

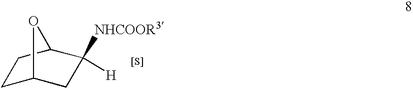

wherein R³ is C₁-C₆ alkyl; and
d) reduction of the carbamate 9 in the presence of lithium aluminium hydride to form the (S)-amine of formula I.

A further embodiment of the invention is the intermediates of formula 6 wherein R is n-butyl.

In a further embodiment the enzymatic reaction (step 5) is carried out with compounds of formula 6 wherein R³ is n-pentyl.

In a further embodiment the enzymatic reaction is carried out in a medium wherein the kosmotropic phosphate anion is employed at pH6-8 at a concentration of 0.01-0.5M, preferably 0.05-0.2M, in combination with a lowered temperature of 0-15° C., preferably around 10° C.

According to the methods described in WO 01/097786 further reaction of compounds of formula I, for example with a compound of formula III yield the adenosine receptor ligands of formula II (Scheme 3). Compounds of formula III can be prepared according to the method described in WO04/00842.

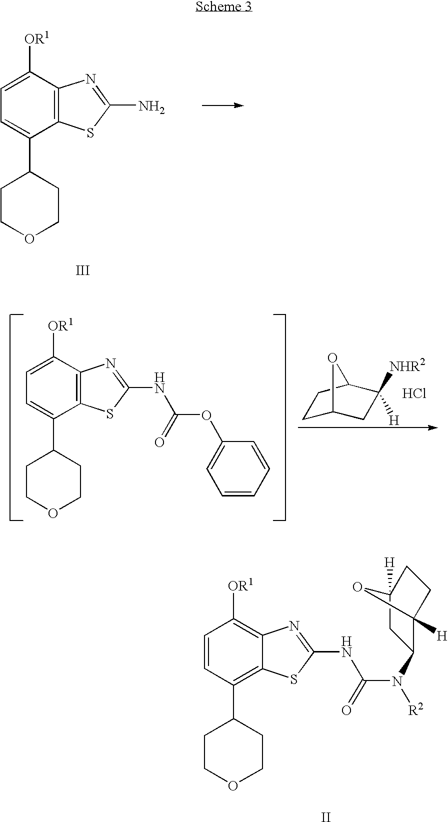

wherein R¹ and R² independently of each other are alkyl.

As used herein, the term “C₁-C₆-alkyl” denotes a saturated straight- or branched-chain alkyl group containing from 1 to 6 carbon atoms, for example, methyl, ethyl, propyl, n-butyl, i-butyl, 2-butyl and the like. Preferred alkyl groups are groups with >4 carbon atoms.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used.

### EXAMPLE 1

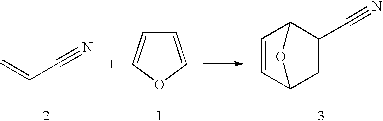

### Preparation of 7-oxabicyclo[2.2.1]hept-5-ene-carbonitrile

In a 1000 mL 4-necked round bottom flask reaction vessel, 77.20 g zinc chloride was added portionwise to 98.00 g (123 mL) acrylonitrile. 381.00 g (408 mL). Furan was added at room temperature, and the reaction mixture was stirred for 8 hours. The resulting solution was diluted with 750 mL ethylacetate and washed twice with 750 mL and 300 mL water. The aqueous phase was back extracted with 500 mL ethylacetate. The organic phases were combined and dried over Na₂SO₄, filtered and concentrated yielding 197.3 g 7-oxabicyclo[2.2.1]hept-5-ene-carbonitrile as a yellow oil.

### EXAMPLE 2

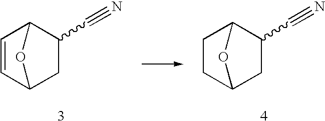

### Preparation of 7-oxabicyclo[2.2.1]heptan-2-carbonitrile

218.0 g 7-oxabicyclo[2.2.1]hept-5-ene-carbonitrile were dissolved in 2.18 L ethylacetate and transferred into a glass reactor under argon atmosphere. 4.36 g Pd/C 10% were added. The reaction mixture was flushed three times with 5 bar H₂ and then hydrogenated at ambient pressure for 24 hours at room temperature. The reaction mixture was filtered, and the solution was concentrated yielding 210.8 g 7-oxa-bicyclo[2.2.1]heptan-2-carbonitrile.

### EXAMPLE 3

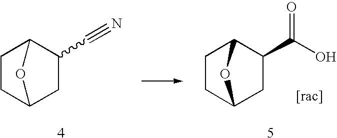

### Preparation of (exo)-7-oxabicyclo[2.2.1]heptane-2-carboxylic acid

A 1500 mL 4-necked round bottom flask was charged with 550 mL ethanol and 200 mL of a 10 M potassium hydroxide. A solution of 100.0 g 7-oxabicyclo[2.2.1]heptan-2-carbonitrile in 250 mL ethanol was added at room temperature. The reaction mixture was refluxed for 1.5 hours, cooled to room temperature and stirred overnight. The ethanol was exchanged with water at constant volume under reduced pressure. Residual ethanol was removed by extraction of the aqueous phase with tert. butylmethyl ether (TBME). The aqueous solution was acidified to pH 1 by addition of 170.2 mL 37% hydrochloric acid and saturated with 60.0 g sodium chloride. The solution was extracted with 4 times with 600 mL TBME. The organic phases were combined, dried over Na₂SO₄, filtered and concentrated yielding 113.0 g (exo)-7-oxabicyclo[2.2.1]heptane-2-carboxylic acid.

### EXAMPLE 4

### Preparation of (exo)-7-oxabicyclo[2.2.1]heptane-2-carboxylic acid butyl ester

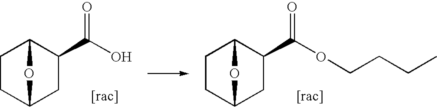

A 1500 mL, 4-necked round bottom flask was charged (under Ar gas) with a solution of 150 g (exo)-7-oxabicyclo[2.2.1]heptane-2-carboxylic acid in 450 mL toluene and 0.6 mL dimethyl formamide. A solution of 97.11 mL oxalyl chloride in 300 mL toluene was added at ambient temperature, the solution was cooled to room temperature and stirred under for one hour. The solvent was evaporated under constant volume by continuously adding 1500 mL toluene. The solution was transferred into a 2500 mL 4-necked round bottom flask, and 77.37 g n-butanol was added to the solution. After stirring for 1 hour at room temperature, a solution of saturated sodium bicarbonate was added to the reaction mixture, which was then filtered to obtain a better separation of the layers. The organic phase was washed with 840 mL deionized water, separated, and the aqueous layer was extracted twice with 225 mL toluene. The organic extracts were combined, washed twice with 10% NaCl-solution, dried with Na₂SO₄ and concentrated. 150 mL. Water was added to the residue. The solution was reconcentrated to remove residual butanol, yielding 180.9 g (exo)-7-oxabicyclo[2.2.1]heptane-2-carboxylic acid butyl ester as a yellow-brown oil.

The corresponding ethyl, propyl, n-pentyl and n-hexyl esters can be prepared by analogy starting with the corresponding alcohols.

### EXAMPLE 5

### Preparation of (1R,2S,4S)-7-oxabicyclo[2.2.1]heptan-2-exo-carboxylic acid

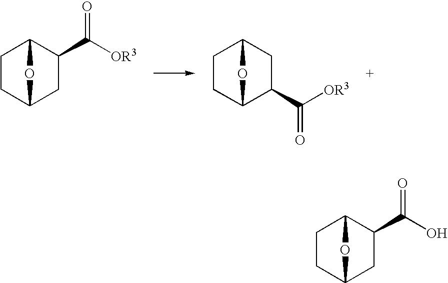

exo-racemate 6 (1S,2R,4R)-6 (6-[R]) (1R,2S,4S)-7
R³=a: n-pentyl b: n-butyl
   a) enzymatic hydrolysis of pentyl ester 6a

95.0 g of pentyl ester 6a (98.1% GC; 439.1 mmol) was emulsified at 28° C. in a mixture of 1.9 L 0.1 M NaCl and 4 mM sodium phosphate buffer pH 7.0 with intensive stirring. The hydrolytic reaction was started by the addition of 19.0 mL Novocor AD L (Novozymes; Denmark), and the pH maintained at 7.0 by the controlled addition (pH-stat) of 1.0 N NaOH solution under intensive stirring at 28° C. After a consumption of 178.4 mL 1.0N NaOH solution (40.6% conversion; 42 hours), the reaction was stopped by adding 1.9 L dichloromethane, and the two phases were allowed to separate. The aqueous phase was washed with 2×1.5 L ethyl acetate, acidified to pH 2.0 (with ca. 29 g 25% HCl) and extracted with 5×1.5 L ethyl acetate. The combined organic phases were dried over Na₂SO₄ and evaporated giving 24.0 g (37%) of (1R,2S,4S)-7-oxabicyclo[2.2.1]heptan-2-exo-carboxylic acid as a white solid. Analytics: GC-purity: 96.2% (silylated); 98.0% ee (column: BGB-175, 15 m×0.25 mm; 70-170 ° C. with 2° C./min; H₂; 50 kPa; Inj.: 200° C.; Det.: 200° C.); [α]_{D}=−29.33° (c=1.05 in EtOH); MS: 143.0 (M+H⁺).

b) enzymatic hydrolysis of butyl ester 6b

20.0 g of butyl ester 6b (98.5% GC; 99.4 mmol) was emulsified at 10° C. in 400 mL 0.1M sodium phosphate buffer pH 7.0 by intensive stirring. The hydrolytic reaction was started by the addition of 3.0 mL Novocor AD L (Novozymes; Denmark) and the pH maintained at 7.0 by the controlled addition (pH-stat) of 1.0 N NaOH solution under intensive stirring at 9-10° C. After 39.9% conversion (96 hours), the reaction was stopped by adding 400 mL dichloromethane and the two phases were allowed to separate. The aqueous phase was washed once more with 400 mL dichloromethane, acidified to pH 1.5 (25% HCl) and extracted with 4×400 mL ethyl acetate. The combined organic phases were dried over sodium sulfate and evaporated giving 5.17 g (36%) of (1R,2S,4S)-7-oxa-bicyclo[2.2.1]heptan-2-exo-carboxylic acid as a white solid. Analytics: GC-purity: 99.3% (silylated); 98.3% ee.

### EXAMPLE 6

### Preparation of [S]-7-oxabicyclo[2.2.1]heptane-2-carboxylic acid ethyl ester

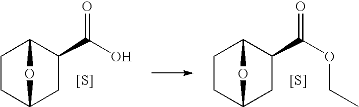

A 1500 mL 4-necked round bottom flask was charged with a solution of 29.00 g 7-oxa-bicyclo[2.2.1]heptane-2-carboxylic acid in 120.0 mL ethanol. 2.4 mL 95% Sulfuric acid was added, the solution was heated at reflux overnight. The solution was then cooled and neutralized with a bicarbonate solution pH 7-8). The resulting suspension was concentrated at 50° C. under reduced pressure. 200 mL TBME and 80 mL water were added. The phases were separated, and the organic phase was washed with water. The aqueous phases were extracted with 50 mL TBME each. The organic phases were combined, dried over Na₂SO₄, filtered and concentrated under reduced pressure yielding 34.03 g 7-oxa-bicyclo[2.2.1]heptane-2-carboxylic acid ethyl ester as a yellow oil.

### EXAMPLE 7

### Preparation of ethyl(7-oxabicyclo[2.2.1]hept-2-yl)-carbamate

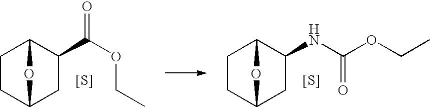

A 1500 mL 4-necked round bottom flask was charged with 20.0 g 7-oxabicyclo[2.2.1]-heptan carboxylic acid ethyl ester. 47.20 g Hydrazine hydrate (24% solution) was added. The reaction mixture was heated to 80° C. and stirred overnight to form the hydrazide. The resulting solution was then cooled to 0° C., 224 mL dichloromethane were added, and subsequently 67.20 g 25% hydrochloric acid was added over a period of 45 minutes. The reaction was exothermic. The reaction mixture was cooled to 0° C., 250.0 g of a 10% sodium nitrite solution was added, and the reaction mixture was stirred for 75 minutes at 0° C. Additional 224 mL dichloromethane was added. The two phases were separated, and the organic phase containing the acylazide was charged into a 1500 mL 4-necked round bottom flask and cooled to −2° C. 50.0 g Sodium sulfate was added, and after stirring the reaction mixture for 15 minutes, 448 mL ethanol was added. The suspension was stirred for 25 minutes at −2° C., then heated to 40 ° C and stirred for 48 hours. The reaction was monitored by HPLC. When no azide could be detected, the suspension was filtered, and the filtrate was concentrated yielding 15.1 g ethyl(7-oxabicyclo[2.2.1]hept-2-yl)-carbamate. The crude product was freed of ethanol by dissolving it in toluene and washing the solution with sodium bicarbonate solution and brine.

### EXAMPLE 8

### Preparation of (7-oxa-bicyclo[2.2.1]hept-2-yl)-amine (I)

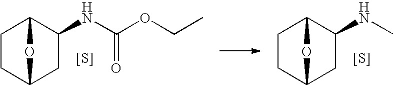

A 500 mL 4-necked round bottom flask was charged with 86 mL 1 molar lithium aluminium hydride. At a temperature of 62° C., a solution of 14.80 g ethyl(7-oxa-bicyclo[2.2.1]hept-2-yl)-carbamate in 60 mL tetrahydrofuran was added over 70 minutes. The reaction was complete after 10 minutes. The reaction mixture was cooled to 0° C. and 9.0 mL of a 0.5 M solution of sodium hydroxide was slowly added. The resulting white suspension was filtered over a pad of dicalite. The filtrate was transferred into a reaction vessel, cooled to 15° C., and 5.50 mL 37% hydrochloric acid was added. The resulting suspension was concentrated to a volume of about 30 mL, and the remaining solvent was replaced by addition of ethyl acetate and subsequent concentration. Finally a thick white suspension was obtained which was filtered. The product was dried at 40° C. under reduced pressure, yielding 9.0 g (7-oxabicyclo[2.2.1]hept-2-yl)-amine. HCl (I).

1. A method for the preparation of a mixture of enantiomerically pure 7 and entaniomerically pure 6-[R] 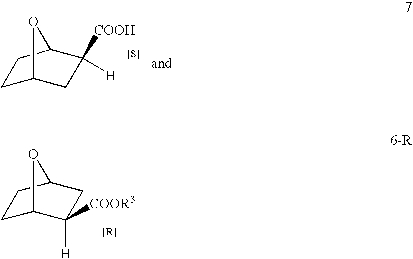
wherein R³ is C₁-C₆-alkyl
which comprises
enzymatically hydrolyzing a racemic exo-ester of formula 6 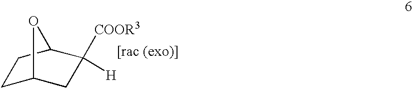
wherein R³ is C₁-C₆-alkyl
in the presence of a lipase to form a mixture of enantiomerically pure 7 and entaniomerically pure 6-[R].

2. The process of claim 1, wherein the lipase is Candida antarctica form A.

3. The process of claim 1, wherein R³ is n-butyl or n-pentyl.

4. The process of claim 1, wherein the reaction is performed in a phosphate buffer at a concentration of 0.01-0.5M.

5. The process of claim 1, wherein the reaction is carried out in a phosphate buffer at a concentration of 0.05-0.2M in combination with a lower temperature.

6. The process of claim 1, wherein the racemic exo-ester of formula 6 is prepared by the process comprising
a) adding acrylonitrile to furan in the presence of a catalytic amount of ZnCl₂ to obtain rac (endo:exo) 7-oxabicyclo[2.2.1]hept-5-ene-2-carbonitrile 3 as a racemic 1:1 mixture of exo/endo-isomers 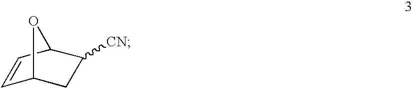
b) catalytically reducing the double bond of rac (endo:exo) 7-oxabicyclo[2.2.1]hept-5-ene-2-carbonitrile 3 in the presence of a metal catalyst to obtain the racemic endo/exo 7-oxabicyclo[2.2.1]heptane-2-carbonitrile 4 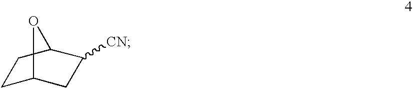
c) hydrolyzing racemic 7-oxabicyclo[2.2.1]heptane-2-carbonitrile 4 in the presence of a strong base in a solvent to obtain exo 7-oxabicyclo[2.2.1]hept-5-ane-2-carboxylic acid 5 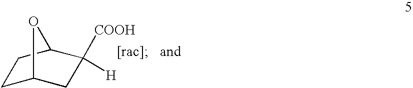
d) esterifying 7-oxabicyclo[2.2.1]hept-5-ane-2-carboxylic acid 5 to form the racemic exo-ester of formula 6.

7. The process of claim 1, further comprising extraction of the mixture to provide a compound of formula 7

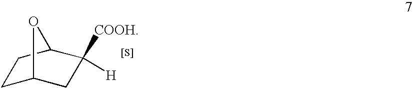

8. The process of claim 1, further comprising extraction of the mixture to provide a compound of formula 6-[R]

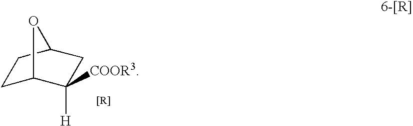

9. A process for the preparation of a compound of formula I 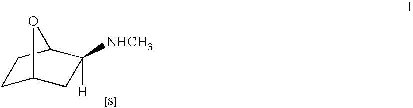
which process comprises
a) adding acrylonitrile to furan in the presence of a catalytic amount of ZnCl₂ to obtain rac (endo:exo) 7-oxabicyclo[2.2.1]hept-5-ene-2-carbonitrile in a racemic 1:1 mixture of exo/endo-isomers 3 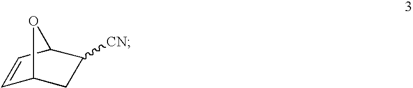
b) catalytically reducing the double bond of rac (endo:exo) 7-oxabicyclo[2.2.1]hept-5-ene-2-carbonitrile in the presence of a metal catalyst to obtain the racemic endo/exo 7-oxa-bicyclo[2.2.1]heptane-2-carbonitrile 4 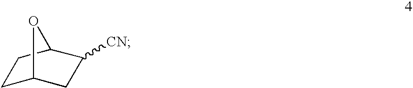
c) hydrolyzing the racemic 7-oxabicyclo[2.2.1]heptane-2-carbonitrile 4 in the presence of a strong base in a solvent to obtain exo 7-oxabicyclo[2.2.1]hept-5-ane-2-carboxylic acid 5 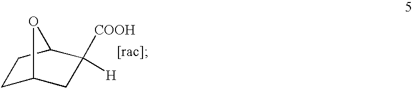
d) esterifying the 7-oxabicyclo[2.2.1]hept-5-ane-2-carboxylic acid 5;
e) enzymatically hydrolyzing the racemic exo-7-oxabicyclo[2.2.1]hept-5-ane-2-carboxylic acid ester of formula 6 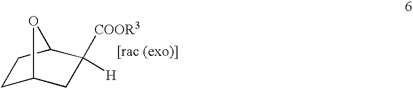
wherein R³ is C₁-C₆-alkyl
in the presence of a lipase to obtain enantiomerically pure (S)-7-oxabicyclo[2.2.1]-heptan-2-exo-carboxylic acid (7) 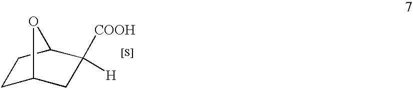
f) esterifying the enantiomerically pure acid 7 to the corresponding (S)-7-oxa-bicyclo[2.2.1]heptane-2-carboxylic acid ethyl ester 8 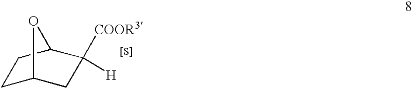
wherein R^{3′} is C₁-C₆-alkyl;
g) transforming carboxylic acid ester 8 by reaction with hydrazine hydrate and subsequently with nitrous acid to form the azide which is rearranged into the carbamic acid ester 9 in the presence of an alkylalcohol 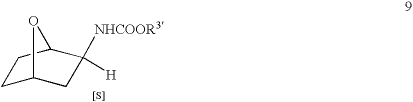
wherein R^{3′} is C₁-C₆-alkyl; and
h) reducing the carbamic acid ester 9 in the presence of lithium aluminium hydride to form the amine of formula I.

10. The compound [exo]-7-oxabicyclo [2.2.1]heptane-2-carboxylic acid butyl ester.

11. A process for the preparation of a compound of formula II 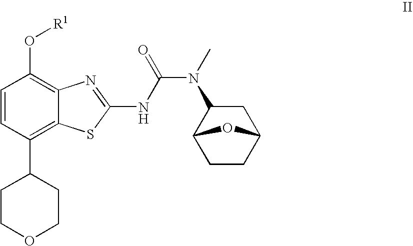
which process comprises reacting a compound of formula I 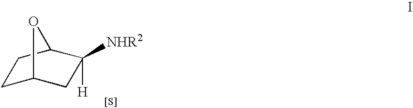
with a compound of formula III 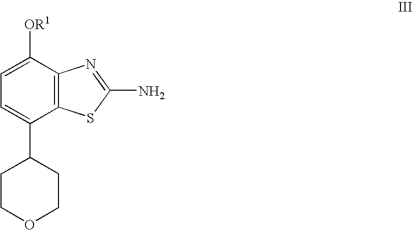
wherein R¹ and R² are each independently C₁₋₆-alkyl.
